(19) United States
(12) Patent Application Publication
(10) Pub. No.: US 2020/0107748 A1
(43) Pub. Date: Apr. 9, 2020

(54) PROBE HAVING MULTIPLE TIPS AND AN INDICATOR FOR OBTAINING BIOELECTRICAL SIGNALS

(71) Applicant: Vine Medical LLC, Saint George, UT (US)

(72) Inventor: Phillip W. Dietz, Saint George, UT (US)

(73) Assignee: Vine Medical LLC, Saint George, UT (US)

(21) Appl. No.: 16/597,494

(22) Filed: Oct. 9, 2019

Related U.S. Application Data
(60) Provisional application No. 62/743,425, filed on Oct. 9, 2018.

(51) Int. Cl. A61B 5/053; A61B 5/00

(52) U.S. Cl. CPC A61B 5/0531; A61B 5/684; A61B 2090/309; A61B 2560/0425; A61B 2562/046; A61B 2562/0209

(57) ABSTRACT

Devices, systems and methods for providing an enhanced bioelectric sensing probe with multiple independent tips and an indicator are disclosed. The device includes an electrical probe having a primary probe tip for measuring bioconductance and one or more secondary probe tips for measuring bioconductance. The electrical probe also includes an indicator. Each tip, whether a primary tip or a secondary tip, is capable of taking an independent bioelectric reading from a patient. The indicator provides feedback regarding the bioelectrical readings. The method includes utilizing the multiple tip sensor head for locating conductance points on a patient.

### CROSS-REFERENCE TO RELATED APPLICATIONS

This application claims the benefit of U.S. Provisional Application No. 62/743,425, filed Oct. 9, 2018, which is incorporated herein by reference in its entirety, including but not limited to those portions that specifically appear hereinafter, the incorporation by reference being made with the following exception: In the event that any portion of the above-referenced provisional application is inconsistent with this application, this application supersedes said above-referenced provisional application.

### TECHNICAL FIELD

The present disclosure relates to obtaining bioelectric information from a patient. In particular, the present disclosure relates to systems and methods for providing and using an enhanced probe with multiple tips and an indicator to facilitate locating and obtaining a bioelectric reading from a patient for assessment, therapeutic and/or diagnostic purposes.

### BACKGROUND

Traditional medical science has long recognized certain electrical characteristics of humans and other living organisms. For example, the traditional medical community has recognized the electrical potentials generated by the human body in such forms as brain waves as detected by electro-encephalographs (EEG), electrical impulses resulting from muscular heart activity as detected by electrocardiograms (EKG), and other electrical potentials measurable at other areas of the human body. While the relative levels of the electrical activity exhibit relatively small levels, such signals are nonetheless measurable and consistent.

In addition to measurable voltage levels, the human body and other mammalian organisms exhibit specific locations on the body wherein the conductance values are relatively predictable for healthy individuals. The locations of anatomical dermal conductance points exhibit a higher conductance value than normal surrounding tissue. Studies have indicated that many conductance points correspond to nerve innervations and trigger points. The conductance points are located under the skin (epidermis) and are accessed electrically through the skin either by the use of needles or by the using a probe tip pressed against the skin. As the outermost layer of epidermis (cornified layer) is less conducive, the probe tip may or may not need a fluid such as water or a type of electrode gel to enhance conductivity through the epidermis to the conductance point.

Interestingly, many of these conductance points match up with traditional acupuncture points. The representative conductance points and their relationship with organs and life systems of the human body have been characterized into more than 800 points that are organized into approximately 14 basic groups or meridians. The measurable state of these conductance points reflects the condition of the related meridians and therefore the health of organs and other functions of the human body. These conductance points are generally located at the extremity region of the hands and feet. The conductance value of normal healthy tissue at a conductance point for different individuals is generally in the same range. In at least some cases, when such tissue is inflamed or infected, the conductivity is higher than the normal range. Additionally, if the tissue is in a degenerative state, the conductivity may be lower.

Systems have been implemented to measure the conductance value at these conductance points and present conductance values to a clinician for use in diagnosing a condition. However, the traditional systems have proven difficult to use since the precise location of the points is difficult to locate and pinpoint. Furthermore, the differences in the characteristics of each patient and each point of a given patient can cause a practitioner to obtain inaccurate and/or unrepeatable readings. Moreover, current technologies have caused discomfort to a patient due to the number of times that a practitioner must take a conductance reading to isolate the correct location of the conductance point.

In some cases, even upon obtaining an accurate measurement at the right location, it can be difficult to correctly determine whether the measurement was accurate. In some systems, a first device is used to locate the points and a second device is brought in contact with the point to perform the conductance testing. While this technique is available, employing multiple devices introduces a potential for clinical error and is very time consuming. In addition, in every case the system used proves difficult to locate the points on the patient. The conductance testing may sometimes be compromised when the system does not accurately determine the points. Also, repeated testing of a certain point may cause the electrical potential of the point to be compromised.

Thus, applicants have recognized that challenges exist in the form of inaccurate readings, unrepeatable readings, pain, discomfort, examination time, and the like. Accordingly, applicant seeks to augment and or replace current devices and techniques.

### SUMMARY

Describe herein is an electrical probe for obtaining bioelectrical signals. The electrical probe includes multiple tips, a primary probe tip, one or more secondary tips, and a verification indicator.

Further described herein is a system for obtaining bioelectrical signals by using a processor and a multi-tip probe. The processor receives a bioelectrical reading from one or more probe tips and determines which bioelectrical reading indicates the highest electrical conductivity among one or more probe tips. Further the processor identifies a direction of the highest electric conductivity among the one or more probe tip from the determined bioelectrical reading and indicates the identified direction by the directional indicator. The probe includes a primary tip, a plurality of secondary probe tips, a directional indicator, and a verification indicator.

Also disclosed is a method for obtaining bioelectrical readings. First, the bioelectrical readings are received by a processer from one or more conductance points. Then the processor decides which bioelectrical reading indicated the highest electrical conductivity. Afterwards the processor transmits the bioelectrical reading to a directional indicator.

### BRIEF DESCRIPTION OF THE DRAWINGS

In order that the manner in which the above recited and other features and advantages of the present disclosure are obtained, a more particular description of the disclosure will be rendered by reference to specific embodiments thereof, which are illustrated in the appended drawings. Understanding that the drawings depict only typical embodiments of the present disclosure and are not, therefore, to be considered as limiting the scope of the disclosure, the present disclosure will be described and explained with additional specificity and detail through the use of the accompanying drawings in which:

FIG. 1 illustrates a perspective side elevation view of a probe;

FIG. 2 illustrates a side view of a probe;

FIG. 3 illustrates a distal view of a probe;

FIG. 4 illustrates a top view of a probe;

FIG. 5 illustrates a distal view of a probe, with a plurality of conductor tips;

FIG. 6 illustrates a perspective side elevation view of a probe with a hood cover:

FIG. 7 illustrates a detailed perspective side elevation view of a probe;

FIG. 8 illustrates a probe contacting a dermal surface layer of a patient;

FIG. 9 illustrates a method of utilizing a probe having multiple tips;

FIG. 10 illustrates an alternative embodiment of a probe:

FIG. 11 illustrates a perspective side elevation view of an alternative embodiment of a probe; and

FIG. 12 illustrates a perspective rear elevation view of the probe of FIG. 11.

### DRAWING-REFERENCE NUMBERS

| | |
|---|---|
| 2 | Probe |
| 4 | Sensor Head |
| 6 | Indicators |
| 8 | Computer Processor |
| 10 | Cord |
| 12 | Housing Unit |
| 14 | Finger Levers |
| 16 | Directional Indicators |
| 18 | Switch |
| 22 | Dermal Surface Layer |
| 24 | Hood |
| 28 | Hood Cover |
| 30 | Primary Probe Tip |
| 32 | Secondary Probe tip |
| 34 | Verification Indicator |

### DETAILED DESCRIPTION

In the following description, for purposes of explanation and not limitation, specific techniques and embodiments are set forth, such as particular techniques and configurations, in order to provide a thorough understanding of the system and device disclosed herein. While the techniques and embodiments will primarily be described in context with the accompanying drawings, those skilled in the art will further appreciate that the techniques and embodiments may also be practiced in other similar devices.

Reference will now be made in detail to the exemplary embodiments, examples of which are illustrated in the accompanying drawings. Wherever possible, the same reference numbers are used throughout the drawings to refer to the same or like parts. It is further noted that elements disclosed with respect to particular embodiments are not restricted to only those embodiments in which they are described. For example, an element described in reference to one embodiment or figure, may be alternatively included in another embodiment or figure regardless of whether or not those elements are shown or described in another embodiment or figure. In other words, elements in the figures may be interchangeable between various embodiments disclosed herein.

The present disclosure relates to obtaining bio-conductance information. In particular, the present disclosure relates to systems and methods for providing and using a probe having multiple tips and an indicator that is used to facilitate locating and obtaining bioelectric reading from a patient for assessment, therapeutic and/or diagnostic purposes.

In the following description of the disclosure, certain terminology is used for the purpose of reference only and is not intended to be limiting. Terms such as “upper,” “lower.” “above,” and “below,” refer to directions in the drawings to which reference is made. Terms such as “inwards” and “outward” refer to directions towards and away from, respectively, the geometric center of the component described. Terms such as “side,” “top,” “bottom,” “horizontal.” “with in,” “distal.” “proximal,” “inside,” and “vertical,” describe the orientation of portions of the component within a consistent but arbitrary frame of reference which is made clear by reference to the text and the associated drawings describing the component under discussion. Such terminology includes words specifically mentioned above, derivatives thereof, and words of similar import.

FIG. 1 illustrates a perspective side elevation view of a probe, which includes probe 2 and housing unit 12. Housing unit 12 provides for a structure to attach together the different components of probe 2. Housing unit 12 may be made from any rigid material such as a wood, glass and or a types of plastic. The shape of the housing unit 12 is such that it fits comfortably in a user's hand. Sensor head 4 is connected to the distal end of probe 2 and is secured to the housing unit by hood 24. Sensor head may have a primary probe tip and one or more secondary probe tips, not shown in FIG. 1. The primary probe tip is located in the center and in the distal end of sensor head 4. One or more secondary probe tips are located to the outer edge of the distal end of sensor head 4 and can be placed at 90° increments. The primary probe tip and one or more of the secondary probe tips interact independently of each other such that each tip may take an independent reading from the patient for measuring and recording the bioelectric reading. Probe 2 is placed on conductance points, is such that they are able to simultaneously or sequentially make contact with the conductance points while obtaining a bioelectric reading from the patient. A conductance point may be a meridian, acupressure or acupuncture point.

In accordance with the conductance data collected by the primary probe tip and the secondary probe tips, a patient's condition may be assessed and/or diagnosed. This assessment and or diagnosis can be aided by using probe 2 to measure the anatomical dermal conductance points located throughout the hands and feet and various other conductance points throughout the body of the patient.

The primary probe tip and one or more secondary probe tips may be shaped in various manners that may include a spherical, conical, cuboidal etc. Moreover, the shape of the primary probe tip may be different that one or more of the secondary tips. Furthermore, the primary probe tip and one or more of the secondary probe tips are made with electrically conductive material that allows for the flow of electricity in one or more directions. This material may include silver, copper, gold, aluminum, zinc, nickel, brass, iron, steel and any other electrically conductive material known to those skilled in the art. The preferred material for both the primary and secondary probe tip is a brass alloy. Both primary probe tip and secondary probe tips may be covered by a hood cover, not shown. This hood cover may be made with non-conductive material to protect the tips from coming in contact with skin. Another embodiment of the tip cover includes apertures that correspond to the primary and one or more of the secondary tips. As the probe is place on the dermal layer of a patient and pressure is placed on finger levers 14 the tips may extend out of the apertures on the tip cover to be able to make contact with the skin. When the pressure is released from finger levers 14 then both primary and secondary probe tips retract bellow the tip cover. Alternatively, another embodiment may include a hood cover that is conductive and allows electricity to flow from both primary and secondary tips.

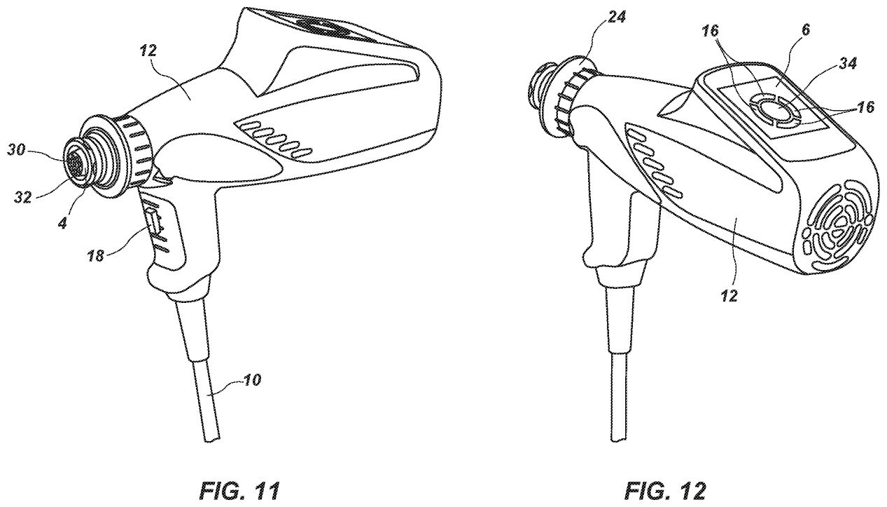

The electrical signal data from the primary probe tip and the secondary probe tips are transmitted to a computer processor not shown. The computer processor receives the electrical signal data and determines whether the primary probe tip or one of the secondary probe tips has the highest bioelectric reading. The computer processor transmits this information to indicator 6. Indicator 6 may include a verification indicator and or directional indicator 16. The verification indicator indicates when the primary probe tip has the highest conductance. The directional indicator indicates when one of the secondary probe tips has the highest conductance. In this embodiment, indicator 6 is located on top of probe 2 near the distal end. Indicator 6 may have one or more directional indicators 16. Directional indicators 16 are typically light-emitting diodes. In some embodiments, the indicator may be a graphical display such as a LCD display as shown in FIGS. 11-12.

The number of directional indicators 16 generally corresponds with the number of secondary tips. In addition, the location of the lights on directional indicator 16 correlates with the location of the secondary tips, not shown, on sensor head 4. When a secondary probe tip is located at the top of sensor head 4, the corresponding directional indicator 16 is located at the top distal end of indicator 6. In contrast, when a secondary probe tip is located on the bottom of sensor head 4, the corresponding directional indicator 16 is located on the bottom of indicator 6. Other directional indicators 16 correspond to secondary probe tips locate on the right or left of the sensor head 4.

While indicator 6 shown demonstrates directional indicator 16, one skilled in the art will recognize that indicator 6 could have several different forms. For example, indicator 6 could be a digital screen or a speaker that vocally gives feedback to the user. In addition, the indicator located anywhere on probe 2, or on other devices that are part of the system.

Sensor head 4 houses both primary and secondary probe tips. To provide electrical power and to relay the data from sensor head 4, probe 2 is connected to cord 10. In an alternative embodiment, probe 2 could be powered by a battery. The data from sensor head 4 could be wirelessly transmitted to a computer processor, including a mobile device tablet or mobile phone, utilizing a system such as Bluetooth. Additionally, data from sensor head 4 may be stored internally to later be transferred to a computer processor. Another embodiment of probe 2 may include a direct connection to a computer processor.

In operation, a user will obtain the device described above. Sensor head 4 is attached to probe 2. The technician or user places sensor head 4, housing both primary and secondary probe tips, on where they believe a meridian or acupressure point is located on the patient. To ensure sensor head 4 is placed firmly against the patient's skin, the user will utilize finger levers 14 located near the distal end of the housing unit 12 are two finger levers 14. Finger levers 14 extend from the housing unit 12 outward. In some configurations, the user will place an index finger on one finger lever 14 and a thumb on the opposing finger lever 14. Generally, the middle finger is available to active the switch 18. In another configuration, the user will place a thumb on finger lever 14 and index finger on the opposing finger lever 14 and the middle finger is used to activate the trigger style switch 18. As the user pushes probe 2 against a patient's body, finger levers 14 assist the user to easily apply the force necessary to operate probe 2.

After the sensor head is appropriately placed the primary probe tip and one or more secondary tips, housed in sensor head 4, obtain bioelectric readings from the patient. Both primary probe tip one or more secondary tips may obtain readings independently of another probe tip. If the highest bioelectric reading is from the primary probe tip then the verification indicator will illuminate. Because the indicator illuminates it is determined that probe 2 is properly located on the patient, and the bioelectric reading is recorded. Probe 2 is then removed from the conductance point. It is also important to note that if the primary probe tip and one or more of the secondary probe tips have a similar or equal high bioelectric reading then the verification indicator and the corresponding directional indicator 16 will illuminate. It is determined that the primary probe tip is at the correct location and the measurement of the bioelectric reading is recorded.

If the highest bioelectric reading is from one of the secondary tips, indicator 6 provides feedback to the user in identifying which a secondary probe tip has the highest reading by illuminating the corresponding directional indicator 16. The user repositions the primary probe tip over or in the direction of the previous location of the secondary probe tip that had the highest bioelectric reading. Probe 2 then obtains another bioelectric reading. If the primary probe tip is now the highest bioelectric reading the verification indicator is illuminated, it is determined that sensor head 4 is properly located, and the bioelectric reading is recorded. Probe 2 is now removed from the meridian or acupressure point. If not, sensor head 4 is readjusted as described above, such that the primary probe tip is placed over or in the direction of the previous location that had the highest bioelectric reading. This process is repeated until the primary probe tip has the highest bioelectric reading.

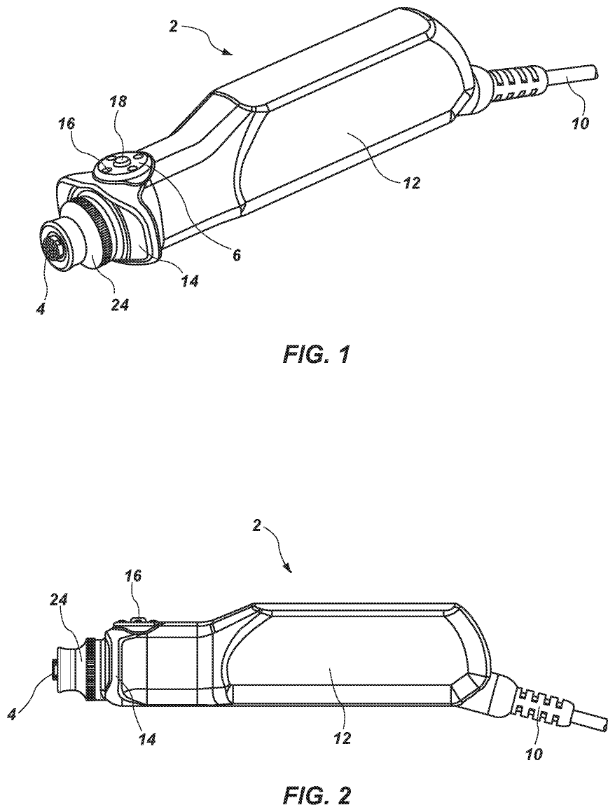

FIG. 2 illustrates a side view of a probe, which includes probe 2 and sensor head 4. Sensor head 4 is connected to the distal end of probe 2. Sensor head 4 may include a primary probe tip and or one or more secondary tips, not shown in FIG. 2. The primary probe tip is located in the center and in the distal end of sensor head 4. One or more secondary probe tips are located to the outer edge of the distal end of sensor head 4 and may be place at 90° increments. The primary probe tip and one or more of the secondary probe tips interact independently of each other such that each tip may take an independent reading from the patient.

The primary probe tip and one or more secondary probe tips may be shaped in various manners that may include a spherical, conical, cuboidal etc. Moreover, the shape of the primary probe tip may be different that one or more of the secondary tips. Furthermore, the primary probe tip and one or more of the secondary probe tips are made with electrically conductive material. This material may include silver, copper, gold, aluminum, zinc, nickel, brass, iron, steel and any other electrically conductive material known to those skilled in the art. The preferred material for both the primary and secondary probe tip is a brass alloy.

The electrical signal data from the primary probe tip and the secondary probe tips are transmitted to a computer processor not shown. The computer processor receives the electrical signal data and determines whether the primary probe tip or one of the secondary probe tips has the highest bioelectric reading. The computer processor transmits this information to indicator 6. Indicator 6 is located on top of probe 2 near the distal end of probe 2. Indicator 6 may have one or more directional indicators 16 and verification indicator. Directional indicators 16 and verification indicators are typically light-emitting diodes. In some embodiments, the indicators may be a graphical display such as a LCD display as shown in FIGS. 11-12.

The number of directional indicators 16 generally corresponds with the number of secondary tips. In addition, the location of the lights on the indicators 6 correlates with the location of the secondary tips, not shown, on sensor head 4. When a secondary probe tip is located at the top of sensor head 4, the corresponding directional indicator 16 is located at the top of indicator 6. In contrast, when a secondary probe tip is located on the bottom of sensor head 4, the corresponding directional indicator 16 is located on the bottom of indicator 6. Other directional indicators 16 correspond to secondary probe tips locate on the right or left of the sensor head 4.

While indicator 6 shown demonstrates directional indicator 16, one skilled in the art will recognize that indicator 6 could have several different forms. For example, indicator 6 could be a digital screen or a speaker that vocally gives feedback to the user. In addition, the indicator located anywhere on probe 2, or on other devices that are part of the system.

To provide electrical power and to relay the data from sensor head 4, probe 2 is connected to cord 10. In an alternative embodiment, probe 2 could be powered by a battery. The data from sensor head 4 could be wirelessly transmitted to a computer processor, including a mobile device tablet or mobile phone, utilizing a system such as Bluetooth. Additionally, data from sensor head 4 may be stored internally to later be transferred to a computer processor. Another embodiment of probe 2 may include a direct connection to a computer processor.

In operation, a user will obtain the device described above. Sensor head 4 is attached to probe 2. The technician or user places sensor head 4 on where they believe a meridian or acupressure point is located on the patient. To ensure sensor head 4 is placed firmly against the patient's skin, the user will utilize finger levers 14 located near the distal end of probe 2. Primary probe tip and one or more secondary tips may be housed in sensor head 4. The primary probe tip and one or more secondary tips each obtain bioelectric readings independently. If the highest bioelectric reading is from the primary probe tip then the verification indicator will illuminate. Because the indicator illuminates it is determined that probe 2 is properly located on the patient, and the bioelectric reading is recorded. Probe 2 is then removed from the meridian or acupressure point. It is also important to note that if the primary probe tip and one or more of the secondary probe tips have a similar or equal high bioelectric reading then the verification indicator and the corresponding directional indicator 16 will illuminate. It is determined that the primary probe tip is at the correct location and the measurement of the bioelectric reading is recorded.

If the highest bioelectric reading is from one of the secondary tips, indicator 6 provides feedback to the user in identifying which secondary probe tip has the highest reading by illuminating the corresponding directional indicator 16. The user repositions the primary probe tip 30 over or in the direction of the previous location of the secondary probe tip that had the highest bioelectric reading. Probe 2 then obtains another bioelectric reading from sensor head 4. If the primary probe tip is now the highest bioelectric reading the verification indicator is illuminated, it is determined that sensor head 4 is properly located, and the bioelectric reading is recorded. Probe 2 is now removed from the conductance point. If not, sensor head 4 is readjusted as described above, such that the primary probe tip is placed over or in the direction of the previous location that had the highest bioelectric reading. This process is repeated until the primary probe tip has the highest bioelectric reading.

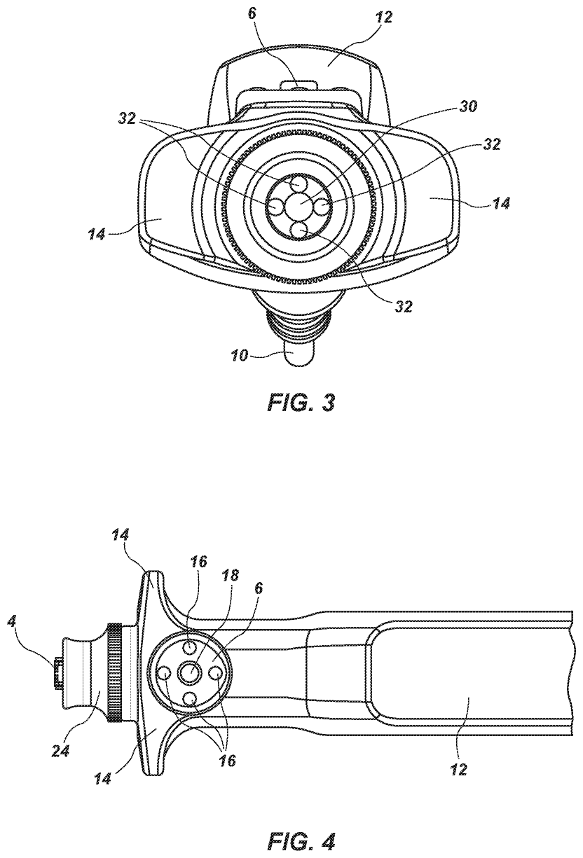

FIG. 3 illustrates a distal view a probe, which includes probe 2. The housing unit 12 provides for a structure to attach the different components of probe 2. The housing unit 12 is made from any rigid material such as wood glass or types of plastic. The shape of the housing unit 12 is such that it fits comfortably in a user's hand and may, for example, resemble a pen substantially contained in a single plane or a pistol shape a grip member extends off the main body in a substantially perpendicular manner. Sensor head 4 is connected to the distal portion of probe 2 and is secured to the housing unit by hood 24. Sensor head may include a primary probe tip and one or more secondary probe tip 32. Primary probe tip 30 is located in the center and in the distal end of sensor head 4. One or more secondary probe tips 32 are located to the outer edge of the distal end of sensor head 4 and may be place at 900 increments. Primary probe tip 30 and one or more of secondary probe tips 32 interact independently of each other such that each tip may take an independent reading from the patient.

Primary probe tip 30 and one or more secondary probe tips 32 may be shaped in various manners that may include a spherical, conical, cuboidal etc. Moreover, the shape of primary probe tip 30 may be different that one or more of secondary probe tips 32. Furthermore, primary probe tip 30 and one or more of secondary probe tips 32 are made with electrically conductive material as in material that allows for the flow of electricity in one or more directions. This material may include silver, copper, gold, aluminum, zinc, nickel, brass, iron, steel and any other electrically conductive material known to those skilled in the art. The preferred material for both primary probe tip 30 and secondary probe tip 32 is a brass alloy. Both primary probe tip 30 and secondary probe tips 32 may be covered by a hood cover, not shown. This hood cover may be made with non-conductive material to protect the tips from coming in contact with skin. Another embodiment of the tip cover includes apertures that correspond to the primary and one or more of secondary probe tips 32. As the prob is place on the dermal layer of a patient the and pressure is placed on finger levers 14 primary probe tip 30 and secondary probe tips 32 may extend out of the apertures on the tip cover to be able to make contact with the skin. When the pressure is released from finger levers 14 then both primary probe tip s 30 and secondary probe tips 32 retract bellow the tip cover.

The electrical signal data from primary probe tip 30 and secondary probe tips 32 are transmitted to a computer processor not shown. The computer processor receives the electrical signal data and determines whether primary probe tip 30 or one of secondary probe tips 32 has the highest bioelectric reading. The computer processor transmits this information to indicator 6. Indicator 6 is located on top of probe 2 near the distal end. Indicator 6 may have one or more directional indicators 16 and a verification indicator. Directional indicators 16 and verification indicators are typically light-emitting diodes. In some embodiments, the indicator may be a graphical display such as a LCD display as shown in FIGS. 11-12.

The number of directional indicators 16 generally corresponds with the number of secondary probe tips 32. In addition, the location of the lights on the indicators 6 correlates with the location of secondary probe tips 32 on sensor head 4. When a secondary probe tip 32 is located at the top of sensor head 4, the corresponding directional indicator 16 is located at the top of indicator 6. In contrast, when secondary probe tip 32 is located on the bottom of sensor head 4, the corresponding directional indicator 16 is located on the bottom of indicator 6. Other directional indicators 16 correspond to secondary probe tips 32 locate on the right or left of the sensor head 4.

While indicator 6 shown demonstrates directional indicator 16, one skilled in the art will recognize that indicator 6 could have several different forms. For example, indicator 6 could be a digital screen or a speaker that vocally gives feedback to the user. In addition, the indicator located anywhere on probe 2, or on other devices that are part of the system.

To provide electrical power and to relay the data from sensor head 4, probe 2 is connected to cord 10. In an alternative embodiment, probe 2 could be powered by a battery. The data from sensor head 4 could be wirelessly transmitted to a computer processor, including a mobile device tablet or mobile phone, utilizing a system such as Bluetooth. Additionally, data from sensor head 4 may be stored internally to later be transferred to a computer processor. Another embodiment of probe 2 may include a direct connection to a computer processor

FIG. 4 illustrates a top view a probe, which includes probe 2. Sensor head 4 is connected to the distal end of probe 2. Sensor head may have a primary probe tip and one or more secondary tip, not shown in FIG. 2. The primary probe tip is located in the center and in the distal end of sensor head 4. One or more secondary probe tips are located to the outer edge of the distal end of sensor head 4 and may be set at 90° increments. The primary probe tip and secondary probe tips interact independently of each other such that each tip may take an independent reading from the patient.

The electrical signal data from the primary probe tip and the secondary probe tips are transmitted to a computer processor not shown. The computer processor receives the electrical signal data and determines whether the primary probe tip or one of the secondary probe tips has the highest bioelectric reading. The computer processor transmits this information to indicator 6. Indicator 6 is located on top of probe 2 near the distal end of probe 2. Indicator 6 may have one or more directional indicators 16. Directional indicators 16 are typically light-emitting diodes. In some embodiments, the indicator may be a graphical display such as a LCD display as shown in FIGS. 11-12.

The number of directional indicators 16 generally corresponds with the number of secondary tips. In addition, the location of the lights on indicators 6 correlates with the location of the primary and secondary tips, not shown, on sensor head 4. When a secondary probe tip is located at the top of sensor head 4, the corresponding directional indicator 16 is located at the top of indicator 6. In contrast, when a secondary probe tip is located on the bottom of sensor head 4, the corresponding directional indicator 16 is located on the bottom of indicator 6. Other directional indicators 16 correspond to secondary probe tips locate on the right or left of the sensor head 4.

While indicator 6 shown demonstrates directional indicator 16, one skilled in the art will recognize that indicator 6 could have several different forms. For example, indicator 6 could be a digital screen or a speaker that vocally gives feedback to the user. In addition, the indicator located anywhere on probe 2, or on other devices that are part of the system.

In operation, a user will obtain the device described above. Sensor head 4 is attached to probe 2. The technician or user places sensor head 4 on where they believe a meridian or acupressure point is located on the patient. To ensure sensor head 4 is placed firmly against the patient's skin, the user will utilize finger levers 14 located near the distal end of probe 2. The primary probe tip and one or more secondary tips each obtain bioelectric readings independently from the patient. If the highest bioelectric reading is from the primary probe tip then the verification indicator will illuminate. Because the indicator illuminates it is determined that probe 2 is properly located on the patient, and the bioelectric reading is recorded. Probe 2 is then removed from the conductance point that may be a meridian or acupressure point. It is also important to note that if the primary probe tip and one or more of the secondary probe tips have a similar or equal high bioelectric reading then the verification indicator and the corresponding directional indicator 16 or verification indicator will illuminate. It is determined that the primary probe tip is at the correct location and the measurement of the bioelectric reading is recorded.

If the highest bioelectric reading is from one of the secondary tips, indicator 6 provides feedback to the user in identifying which a secondary probe tip has the highest reading by illuminating the corresponding directional indicator 16. The user repositions the primary probe tip 30 over or in the direction of the previous location of the secondary probe tip that had the highest bioelectric reading. Probe 2 obtains another bioelectric reading. If the primary probe tip is now the highest bioelectric reading the verification indicator is illuminated, it is determined that sensor head 4 is properly located, and the bioelectric reading is recorded. Probe 2 is now removed from the meridian or acupressure point. If not, sensor head 4 is readjusted as described above, such that the primary probe tip is placed over or in the direction of the previous location that had the highest bioelectric reading. This process is repeated until the primary probe tip has the highest bioelectric reading.

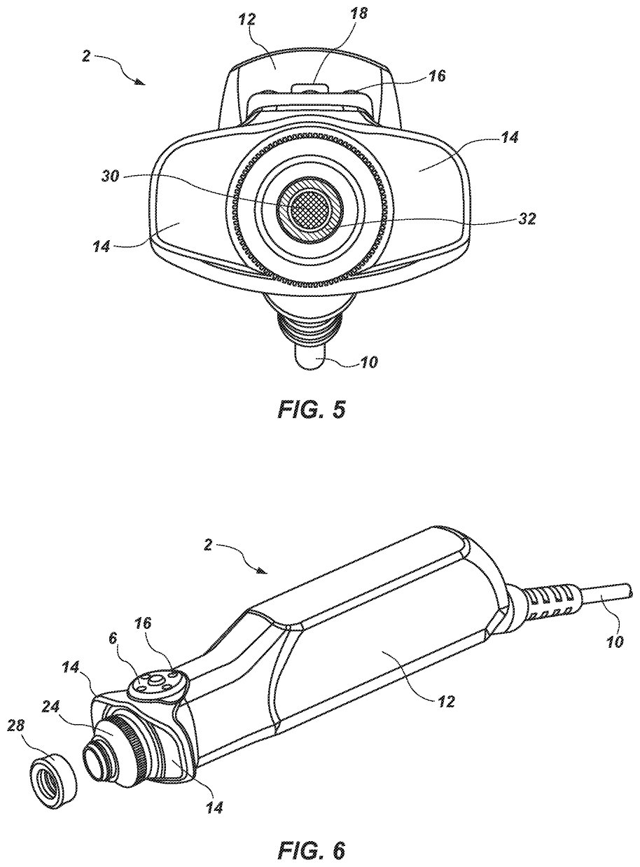

FIG. 5 illustrates a distal view of a probe, which includes probe 2. The sensor head is connected to the distal end of probe 2. Sensor head may have a primary probe tip and one or more secondary tip, not shown in FIG. 2. Primary probe tip 30 is located in the center and in the distal end of the sensor head. One or more secondary probe tips are located to the outer edge of the distal end of the sensor head. Secondary probe tips 32 may be placed at 90° increments around the outer edge of the sensor head or it may be a ring around the outer edge of the sensor head. Primary probe tip 30 and one or more of secondary probe tips 32 interact independently of each other such that each tip may take an independent reading from the patient.

Primary probe tip 30 and one or more secondary probe tips 32 may be shaped in various manners that may include a spherical, conical, cuboidal, ring shaped etc. Moreover, the shape of primary probe tip 30 may be different that one or more of secondary probe tips 32. Furthermore, primary probe tip 30 and one or more of secondary probe tips 32 are made with electrically conductive material that allows for the flow of electricity in one or more directions. This material may include silver, copper, gold, aluminum, zinc, nickel, brass, iron, steel and any other electrically conductive material known to those skilled in the art. The preferred material for both primary probe tip 30 and secondary probe tip 32 is a brass alloy.

The electrical signal data from primary probe tip 30 and secondary probe tips 32 are transmitted to a computer processor not shown. The computer processor receives the electrical signal data and determines whether primary probe tip 30 or one of secondary probe tips 32 has the highest bioelectric reading. The computer processor transmits this information to indicator 6. Indicator 6 is located on top of probe 2 near the distal end of probe 2. Indicator 6 may have one or more directional indicators 16. Directional indicators 16 are typically light-emitting diodes. In some embodiments, the indicator may be a graphical display such as a LCD display as shown in FIGS. 11-12.

The number of directional indicators 16 generally corresponds with the number of secondary probe tips 32. In addition, the location of the lights on the indicators 6 correlates with the location of secondary probe tips 32 on the sensor head. When secondary probe tip 32 is located at the top of the sensor head, the corresponding directional indicator 16 is located at the top of indicator 6. In contrast, when secondary probe tip 32 is located on the bottom of the sensor head, the corresponding directional indicator 16 is located on the bottom of indicator 6. Other directional indicators 16 correspond to secondary probe tips 32 locate on the right or left of the sensor head.

While indicator 6 shown demonstrates directional indicator 16, one skilled in the art will recognize that indicator 6 could have several different forms. For example, indicator 6 could be a digital screen or a speaker that vocally gives feedback to the user. In addition, the indicator located anywhere on probe 2, or on other devices that are part of the system.

To provide electrical power and to relay the data from the sensor head, probe 2 is connected to cord 10. In an alternative embodiment, probe 2 could be powered by a battery. The data from the sensor head could be wirelessly transmitted to a computer processor, including a mobile device tablet or mobile phone, utilizing a system such as Bluetooth. Additionally, data from the sensor head may be stored internally to later be transferred to a computer processor. Another embodiment of probe 2 may include a direct connection to a computer processor

In operation, a user will obtain the device described above. The sensor head is attached to probe 2. The technician or user places the sensor head on where they believe a conductance point is located on the patient. To ensure the sensor head is placed firmly against the patient's skin, the user will utilize finger levers 14 located near the distal end of probe 2. Primary probe tip 30 and one or more secondary probe tips 32, each obtain bioelectric readings independently from the patient. If the highest bioelectric reading is from primary probe tip 30 then the verification indicator, not shown, will illuminate. Because the indicator illuminates it is determined that probe 2 is properly located on the patient, and the bioelectric reading is recorded. Probe 2 is then removed from the meridian or acupressure point. It is also important to note that if primary probe tip 30 and one or more of secondary probe tips 32 have a similar or equal high bioelectric reading then the verification indicator and the corresponding directional indicator 16 will illuminate. It is determined that primary probe tip 30 is at the correct location and the measurement of the bioelectric reading is recorded.

If the highest bioelectric reading is from one of secondary probe tips 32, indicator 6 provides feedback to the user in identifying which a secondary probe tip 32 has the highest reading by illuminating the corresponding directional indicator 16. The user repositions primary probe tip 30 over or in the direction of the previous location of secondary probe tip 32 that had the highest bioelectric reading. Probe 2 then obtains another bioelectric reading. If primary probe tip 30 is now the highest bioelectric reading the verification indicator is illuminates, and it is determined that the sensor head is properly located, and the bioelectric reading is recorded. Probe 2 is now removed from the meridian or acupressure point. If not, the sensor head is readjusted as described above, such that primary probe tip 30 is placed over or in the direction of the previous location that had the highest bioelectric reading. This process is repeated until primary probe tip 30 has the highest bioelectric reading.

FIG. 5 illustrates only one secondary tip having a spherical shape surrounding the primary tip. With this configuration, a user can verify that the test location is properly located or determined if the primary tip has the highest reading, but direction cannot be determined in the event that the secondary tip has the highest reading.

FIG. 6 illustrates a perspective side elevation view of a probe, which includes probe 2. The sensor head is connected to the distal end of probe 2. Sensor head may include a primary probe tip and or one or more secondary tip, not shown in FIG. 2. The primary probe tip is located in the center and in the distal end of the sensor head. One or more secondary probe tips are located to the outer edge of the distal end of the sensor head. The primary probe tip and one or more of the secondary probe tips interact independently of each other such that each tip may take an independent reading from the patient.

The primary probe tip and one or more secondary probe tips may be shaped in various manners that may include a spherical, conical, cuboidal etc. Moreover, the shape of the primary probe tip may be different that one or more of the secondary tips. Furthermore, the primary probe tip and one or more of the secondary probe tips are made with electrically conductive material. This material may include silver, copper, gold, aluminum, zinc, nickel, brass, iron, steel and any other electrically conductive material known to those skilled in the art. The preferred material for both the primary and secondary probe tip is a brass alloy. Both primary probe tip and secondary probe tips may be covered by hood cover 28 This hood cover may be made with non-conductive material to protect the tips from inadvertent contact with skin. Another embodiment of tip cover 28 includes apertures that correspond to the primary and one or more of the secondary tips. As the prob is place on the dermal layer of a patient the and pressure is placed on finger levers 14 the tips may extend out of the apertures on tip cover 28 to be able to make contact with the skin. When the pressure is released from finger levers 14 then both primary and secondary probe tips retract bellow tip cover 28. Another embodiment of tip cover 28 may include a conductive tip cover 28 that allows both primary prob and secondary probe(s) to connect to the skin of a patient without being removed.

The electrical signal data from the primary probe tip and the secondary probe tips are transmitted to a computer processor not shown. The computer processor receives the electrical signal data and determines whether the primary probe tip or one of the secondary probe tips has the highest bioelectric reading. The computer processor transmits this information to indicator 6. Indicator 6 is located on top of probe 2 near the distal end of probe 2. Indicator 6 may have one or more directional indicators 16. Directional indicators 16 are typically light-emitting diodes. In some embodiments, the indicator may be a graphical display such as a LCD display as shown in FIGS. 11-12.

The number of directional indicators 16 generally corresponds with the number of secondary tips. In addition, the location of the lights on the indicators 6 correlates with the location of the secondary tips, not shown, on the sensor head. When a secondary probe tip is located at the top of the sensor head, the corresponding directional indicator 16 is located at the top of indicator 6. In contrast, when a secondary probe tip is located on the bottom of the sensor head, the corresponding directional indicator 16 is located on the bottom of indicator 6. Other directional indicators 16 correspond to secondary probe tips locate on the right or left of the sensor head.

While indicator 6 shown demonstrates directional indicator 16, one skilled in the art will recognize that indicator 6 could have several different forms. For example, indicator 6 could be a digital screen or a speaker that vocally gives feedback to the user. In addition, the indicator located anywhere on probe 2, or on other devices that are part of the system.

To provide electrical power and to relay the data from the sensor head, probe 2 is connected to cord 10. In an alternative embodiment, probe 2 could be powered by a battery. The data from the sensor head could be wirelessly transmitted to a computer processor, including a mobile device tablet or mobile phone, utilizing a system such as Bluetooth. Additionally, data from sensor head may be stored internally to later be transferred to a computer processor. Another embodiment of probe 2 may include a direct connection to a computer processor.

In operation, a user will obtain the device described above. The sensor head is attached to probe 2. The technician or user places the sensor head on where they believe a meridian or acupressure point is located on the patient. To ensure the sensor head is placed firmly against the patient's skin, the user will utilize finger levers 14 located near the distal end of probe 2. The primary probe tip and one or more secondary tips, not shown, each obtain bioelectric readings independently from the patient. If the highest bioelectric reading is from the primary probe tip then the verification indicator, not shown, will illuminate. Because the indicator illuminates it is determined that probe 2 is properly located on the patient, and the bioelectric reading is recorded. Probe 2 is then removed from the meridian or acupressure point (conductance point). It is also important to note that if the primary probe tip and one or more of the secondary probe tips have a similar or equal high bioelectric reading then the verification indicator and the corresponding directional indicator 16 will illuminate. It is determined that the primary probe tip is at the correct location and the measurement of the bioelectric reading is recorded.

If the highest bioelectric reading is from one of the secondary tips, indicator 6 provides feedback to the user in identifying which a secondary probe tip has the highest reading by illuminating the corresponding directional indicator 16. The user repositions the primary probe tip 30 over or in the direction of the previous location of the secondary probe tip that had the highest bioelectric reading. Probe 2 obtains another bioelectric reading. If the primary probe tip is now the highest bioelectric reading the verification indicator is illuminated, it is determined that the sensor head is properly located, and the bioelectric reading is recorded. Probe 2 is now removed from the meridian or acupressure point. If not, the sensor head is readjusted as described above, such that the primary probe tip is placed over or in the direction of the previous location that had the highest bioelectric reading. This process is repeated until the primary probe tip has the highest bioelectric reading.

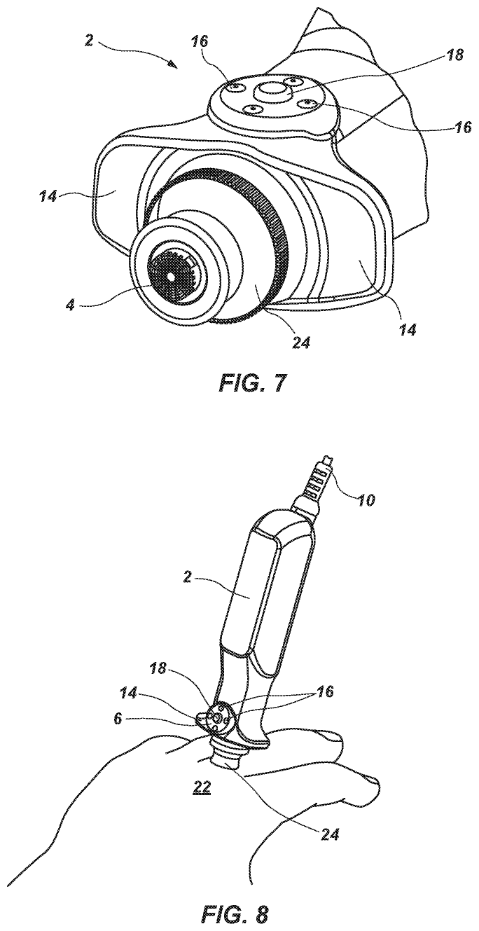

FIG. 7 illustrates a detailed perspective side elevation view of a probe, which includes probe 2. Sensor head 4 is connected to the distal end of probe 2. Sensor head may have a primary probe tip and or one or more secondary tip. The primary probe tip is located in the center and in the distal end of sensor head 4. One or more secondary probe tips are located to the outer edge of the distal end of sensor head 4 and may be place at 90° increments. The primary probe tip and one or more of the secondary probe tips interact independently of each other such that each tip may take an independent reading from the patient.

The electrical signal data from the primary probe tip and the secondary probe tips are transmitted to a computer processor not shown. The computer processor receives the electrical signal data and determines whether the primary probe tip or one of the secondary probe tips has the highest bioelectric reading. The computer processor transmits this information to indicator 6. Indicator 6 is located on top of probe 2 near its distal end. Indicator 6 may have one or more directional indicators 16. Directional indicators 16 are typically light-emitting diodes. In some embodiments, the indicator may be a graphical display such as a LCD display as shown in FIGS. 11-12.

The number of directional indicators 16 generally corresponds with the number of secondary tips. In addition, the location of the lights on the indicators 6 correlates with the location of the secondary tips on sensor head 4. When a secondary probe tip is located at the top of sensor head 4, the corresponding directional indicator 16 is located at the top of indicator 6. In contrast, when a secondary probe tip is located on the bottom of sensor head 4, the corresponding directional indicator 16 is located on the bottom of indicator 6. Other directional indicators 16 correspond to secondary probe tips locate on the right or left of sensor head 4.

While indicator 6 shown demonstrates directional indicator 16, one skilled in the art will recognize that indicator 6 could have several different forms. For example, indicator 6 could be a digital screen or a speaker that vocally gives feedback to the user. In addition, the indicator located anywhere on probe 2, or on other devices that are part of the system.

In operation, a user will obtain the device described above. Sensor head 4 is attached to probe 2. The technician or user places sensor head 4 on where they believe a meridian or acupressure point is located on the patient. To ensure sensor head 4 is placed firmly against the patient's skin, the user will utilize finger levers 14 located near the distal end of probe 2. The primary probe tip and one or more secondary tips each obtain bioelectric readings independently from the patient. If the highest bioelectric reading is from the primary probe tip then the verification indicator will illuminate. Because the indicator illuminates it is determined that probe 2 is properly located on the patient, and the bioelectric reading is recorded. Probe 2 is then removed from the meridian or acupressure point. It is also important to note that if the primary probe tip and one or more of the secondary probe tips have a similar or equal high bioelectric reading then the verification indicator and the corresponding directional indicator 16 will illuminate. It is determined that the primary probe tip is at the correct location and the measurement of the bioelectric reading is recorded.

If the highest bioelectric reading is from one of the secondary tips, indicator 6 provides feedback to the user in identifying which a secondary probe tip has the highest reading by illuminating the corresponding directional indicator 16. The user repositions the primary probe tip 30 over or in the direction of the previous location of the secondary probe tip that had the highest bioelectric reading. Probe 2 obtains another bioelectric reading. If the primary probe tip is now the highest bioelectric reading the verification indicator is illuminated, it is determined that sensor head 4 is properly located, and the bioelectric reading is recorded. Probe 2 is now removed from the meridian or acupressure point. If not, sensor head 4 is readjusted as described above, such that the primary probe tip is placed over or in the direction of the previous location that had the highest bioelectric reading. This process is repeated until the primary probe tip has the highest bioelectric reading.

FIG. 8 illustrates a probe contacting a dermal surface layer of a patient, which includes probe 2. The sensor head is connected to the distal end of probe 2. Sensor head may have a primary probe tip and or one or more secondary tip, not shown in FIG. 2. The primary probe tip is located in the center and in the distal end of the sensor head. One or more secondary probe tips are located to the outer edge of the distal end of the sensor head. The primary probe tip and one or more of the secondary probe tips interact independently of each other such that each tip may take an independent reading from the patient.

The electrical signal data from the primary probe tip and the secondary probe tips are transmitted to a computer processor not shown. The computer processor receives the electrical signal data and determines whether the primary probe tip or one of the secondary probe tips has the highest bioelectric reading. The computer processor transmits this information to indicator 6. Indicator 6 is located on top of probe 2 near the distal end of probe 2. Indicator 6 may have one or more directional indicators 16. Directional indicators 16 are typically light-emitting diodes. In some embodiments, the indicator may be a graphical display such as a LCD display as shown below in FIGS. 11-12.

The number of directional indicators 16 generally corresponds with the number of secondary tips. In addition, the location of the lights on the indicators 6 correlates with the location of the secondary tips, not shown, on sensor head 4. When a secondary probe tip is located at the top of sensor head 4, the corresponding directional indicator 16 is located at the top of indicator 6. In contrast, when a secondary probe tip is located on the bottom of sensor head 4, the corresponding directional indicator 16 is located on the bottom of indicator 6. Other directional indicators 16 correspond to secondary probe tips locate on the right or left of the sensor head 4.

While indicator 6 shown demonstrates directional indicator 16, one skilled in the art will recognize that indicator 6 could have several different forms. For example, indicator 6 could be a digital screen or a speaker that vocally gives feedback to the user. In addition, the indicator located anywhere on probe 2, or on other devices that are part of the system.

To provide electrical power and to relay the data from sensor head 4, probe 2 is connected to cord 10. In an alternative embodiment, probe 2 could be powered by a battery. The data from sensor head 4 could be wirelessly transmitted to a computer processor, including a mobile device tablet or mobile phone, utilizing a system such as Bluetooth. Additionally, data from sensor head 4 may be stored internally to later be transferred to a computer processor. Another embodiment of probe 2 may include a direct connection to a computer processor

In operation, a user will obtain the device described above. Sensor head 4 is attached to probe 2. The technician or user places sensor head 4 on where they believe a meridian or acupressure point is located on the patient. To ensure sensor head 4 is placed firmly against the patient's skin, the user will utilize finger levers 14 located near the distal end of the housing unit 12 are two finger levers 14. Finger levers 14 extend from the housing unit 12 outward. In some configurations, the user will place an index finger on one finger lever 14 and a thumb on the opposing finger lever 14. Generally, the middle finger is available to active the switch 18. In another configuration, the user will place a thumb on finger lever 14 and index finger on the opposing finger lever 14 and the middle finger to activate the trigger style switch 18. As the user pushes probe 2 against a patient's body, finger levers 14 assist the user to easily apply the force necessary to operate probe 2. The primary probe tip and one or more secondary tips, not shown, each obtain bioelectric readings independently from the patient. If the highest bioelectric reading is from the primary probe tip then the verification indicator, not shown, will illuminate. Because the indicator illuminates it is determined that probe 2 is properly located on the patient, and the bioelectric reading is recorded. Probe 2 is then removed from the meridian or acupressure point. It is also important to note that if the primary probe tip and one or more of the secondary probe tips have a similar or equal high bioelectric reading then the verification indicator and the corresponding directional indicator 16 will illuminate. It is determined that the primary probe tip is at the correct location and the measurement of the bioelectric reading is recorded.

If the highest bioelectric reading is from one of the secondary tips, indicator 6 provides feedback to the user in identifying which a secondary probe tip has the highest reading by illuminating the corresponding directional indicator 16. The user repositions the primary probe tip 30 over or in the direction of the previous location of the secondary probe tip that had the highest bioelectric reading. Probe 2 obtains another bioelectric reading. If the primary probe tip is now the highest bioelectric reading the verification indicator is illuminated, it is determined that sensor head 4 is properly located, and the bioelectric reading is recorded. Probe 2 is now removed from the meridian or acupressure point. If not, sensor head 4 is readjusted as described above, such that the primary probe tip is placed over or in the direction of the previous location that had the highest bioelectric reading. This process is repeated until the primary probe tip has the highest bioelectric reading.

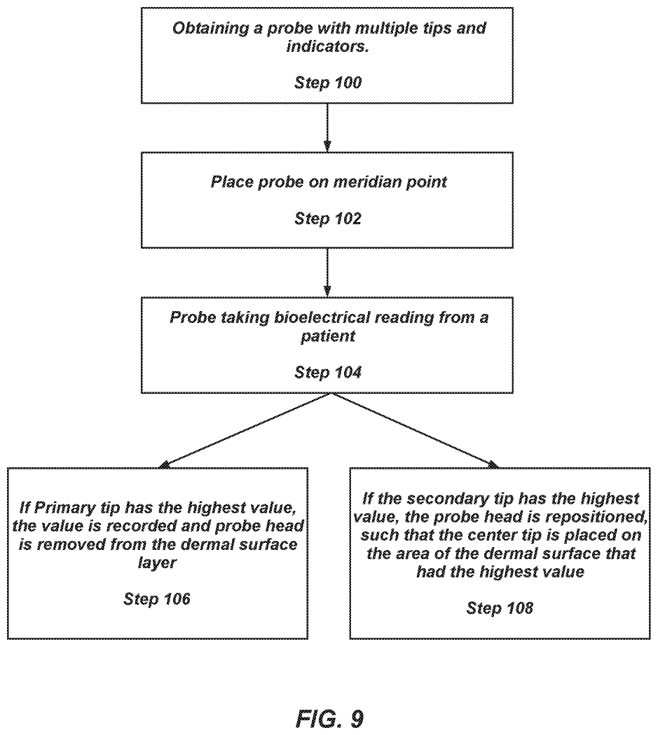

FIG. 9 illustrates a method of utilizing a probe having multiple tips, which includes obtaining a probe with multiple tips and indicators 100. Then the multiple tip probe is placed on a meridian or acupuncture point 200. An electrical pulse is then sent out and the probe taking the bioelectrical reading from the patient 104. The bioelectrical reading is evaluated according to what tip has the highest value. If the primary probe tip has the highest value, the value is recorded, and the probe head is removed from the dermal surface layer of the patient 106. On the other hand, if the secondary probe tip has the highest value, the probe head is repositioned, such that the center of the tip is place on the area of the dermal surface that had the highest value 108. Then if the primary probe tip has the highest value, the value is recorded, and the probe head is removed from the dermal surface layer of the patientn 104. If the secondary probe tip has the highest value repeat step 108.

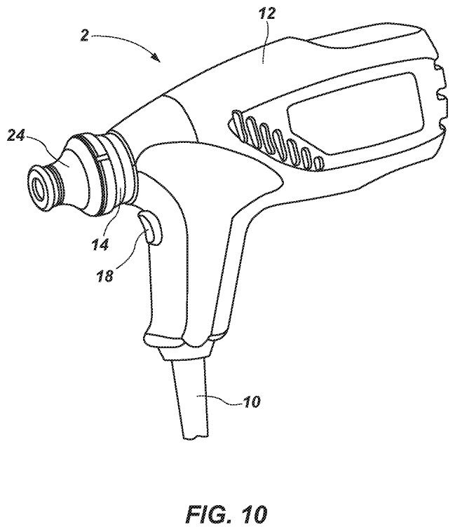

FIG. 10 illustrates an alternative embodiment of a probe, which includes housing unit 12 that is pistol shaped where probe 2 may be griped by holding a grip member that extends off the main body of probe 2 and is substantially perpendicular to the main body. Substantially perpendicular is to mean between 55 and 90°. The switch 18 is placed on the grip handle for easy access and cord 10 extends off the grip member. Alternatively probe 2 may alternatively be powered by a battery. Similar to the other embodiments the hood 24 is located between finger lever 14 and sensor head. Hood may function to connect sensor head to finger lever 14 or may be used to adjust the length of the primary probe tip and or the secondary tip(s). Furthermore, finger levers are place on probe 2 between hood 24 and housing unit 12. This embodiment of the device functions substantially similar to the embodiment described in FIG. 1 with the exception of the placement of the hands on the device.

FIGS. 11-12 illustrate an alternative embodiment of a probe, which includes housing unit 12 that is pistol shaped where probe that includes a grip member that extends of the main part of housing unit 12. Trigger style switch 18 may be placed on the grip member to allow for easy access when gripping the probe. Generally, the middle finger is available to active the switch 18. In another configuration, the user will place a thumb on finger lever 14 and index finger on the opposing finger lever 14 and the middle finger to activate the trigger style switch 18. As the user pushes probe 2 against a patient's body, finger levers 14 assist the user to easily apply the force necessary to operate probe 2. Extending out the bottom end of the grip member is cord 10. Alternatively, cord may extend out of housing 12 at the opposite end of sensor head 4. Hood 14 is located in between sensor head 4 and housing 12. Sensor head includes primary probe tip 30 and one or more secondary tip(s) 32.

The electrical signal data from primary probe tip 30 and secondary probe tips 32 are transmitted to a computer processor not shown. The computer processor receives the electrical signal data and determines whether primary probe tip 30 or one of secondary probe tips 32 has the highest bioelectric reading. The computer processor transmits this information to indicator 6. Indicator 6 is located on top of probe 2 near the distal end of probe 2. Indicator 6 may have one or more directional indicators 16. Directional indicators 16 are typically light-emitting diodes. In some embodiments, the indicator may be a graphical display such as a LCD display as shown.

The number of directional indicators 16 generally corresponds with the number of secondary probe tips 32. In addition, the location of the lights on the indicators 6 correlates with the location of secondary probe tips 32 on the sensor head. When secondary probe tip 32 is located at the top of the sensor head, the corresponding directional indicator 16 is located at the top of indicator 6. In contrast, when a secondary probe tip 32 is located on the bottom of the sensor head, the corresponding directional indicator 16 is located on the bottom of indicator 6. Other directional indicators 16 correspond to secondary probe tips 32 locate on the right or left of the sensor head.

While indicator 6 shown demonstrates directional indicator 16, one skilled in the art will recognize that indicator 6 could have several different forms. For example, indicator 6 could be a digital screen or a speaker that vocally gives feedback to the user. In addition, the indicator located anywhere on probe 2, or on other devices that are part of the system. This embodiment of the device functions substantially similar to the embodiment described in FIG. 1 with the exception of the placement of the hands on the device.

### Examples

Example 1 is an electrical probe that includes a primary probe tip for measuring bioconductance; one or more secondary probe tips for measuring bioconductance; and an indicator.

Example 2 is the electrical probe of Example 1, wherein the one or more secondary probe tips are disposed around the primary probe tip at 90° increments.

Example 3 is the electrical probe of any of Examples 1-2, wherein the primary tip and one or more secondary tips are made of a conductive material.

Example 4 is the electrical probe of any of Examples 1-3, wherein the indicator is implemented as a light emitting diode.

Example 5 is the electrical probe of any of Examples 1-4, wherein the indicator is a directional indicator.

Example 6 is the electrical probe of any of Examples 1-5, wherein the directional indicator indicates which of the one or more secondary probe tips provides a highest bioelectrical reading.

Example 7 is the electrical probe of any of Examples 1-6, wherein the directional indicator is implemented as one or more lights which are disposed at 90° increments around a circle.

Example 8 is the electrical probe of any of Examples 1-7, wherein the directional indicator indicates a direction the probe is to be moved to locate a conductance point with the primary probe tip.

Example 9 is the electrical probe of any of Examples 1-8, wherein the directional indicator is implemented as an LCD screen.

Example 10 is the electrical probe of any of Examples 1-9, wherein the directional indicator includes one or more lights associated with each of the one or more secondary probe tips.

Example 11 is the electrical probe of any of Examples 1-10, wherein the probe further includes a grip member that is connected to the probe and is positioned to be substantially perpendicular to the primary probe tip.

Example 12 is the electrical probe of any of Examples 1-11, wherein the probe is connected to an electrical cord.

Example 13 is the electrical probe of any of Examples 1-12, wherein the probe includes a battery.

Example 14 is the electrical probe of any of Examples 1-13, wherein the indicator is located on the distal end of the probe.

Example 15 is the electrical probe of any of Examples 1-14, wherein the indicator is a verification indicator.

Example 16 is the electrical probe of any of Examples 1-15, wherein the one or more secondary probe tips are disposed around the primary probe tip at an angle increment that falls within a range of 10° to 90° increments.

Example 17 is the electrical probe of any of Examples 1-16, wherein the one or more secondary probe tips are disposed around the primary probe tip at an angle increment that falls within a range of 10° to 180° increments.

Example 18 is a system that includes a processor that receives a bioelectrical reading from one or more probe tips, and determines which bioelectrical reading from the one or more probe tips indicates a highest electrical conductivity among the one or more probe tips. The system further includes a probe that includes a primary probe tip, one or more secondary probe tips, a directional indicator, and a verification indicator, wherein the processor further identifies a direction of the highest electrical conductivity among the one or more secondary probe tips for the primary probe tip from the determined bioelectrical reading and indicates the identified direction by the directional indicator.

Example 19 is the system of Example 18, wherein the directional indicator comprises one or more light emitting diodes.

Example 20 is a method that includes receiving, by one or more processors, a bioelectrical reading from one or more secondary probe tips; determining, by the one or more processors, which bioelectrical reading from the one or more secondary probe tips indicates the highest electrical conductivity; identifying, by the one or more processors, a direction of the bioelectrical reading from the one or more probe tips for a primary probe tip; displaying, by the one or more processors, a directional indicator associated with the identified direction.

Example 21 includes the method of Example 20, wherein the receiving of the bioelectrical reading from one or more secondary probe tips occurs in response to placing one or more of the secondary probe tips in contact with person's skin.

Example 22 includes the method of any of Examples 20-21, wherein displaying the directional indicator includes one of illuminating a directional indicator and providing a directional indicator on a screen.

The foregoing description has been presented for the purposes of illustration and description. It is not intended to be exhaustive or to limit the disclosure to the precise form disclosed. Many modifications and variations are possible in light of the above teaching. Further, it should be noted that any or all of the aforementioned alternate implementations may be used in any combination desired to form additional hybrid implementations of the disclosure.

Further, although specific implementations of the disclosure have been described and illustrated, the disclosure is not to be limited to the specific forms or arrangements of parts so described and illustrated. The scope of the disclosure is to be defined by the claims appended hereto, any future claims submitted here and in different applications, and their equivalents.

What is claimed is:

1. An electrical probe comprising:
a primary probe tip for measuring bioconductance;
one or more secondary probe tips for measuring bioconductance; and
an indicator.

2. The electrical probe of claim 1, wherein the one or more secondary probe tips are disposed around the primary probe tip at 90° increments.

3. The electrical probe of claim 1, wherein the primary tip and one or more secondary tips are made of a conductive material.

4. The electrical probe in claim 1, wherein the indicator is implemented as a light emitting diode.

5. The electrical probe of claim 1, wherein the indicator is a directional indicator.

6. The electrical probe of claim 5, wherein the directional indicator indicates which of the one or more secondary probe tips provides a highest bioelectrical reading.

7. The electrical probe of claim 5, wherein the directional indicator is implemented as one or more lights which are disposed at 90° increments around a circle.

8. The electrical probe of claim 5, wherein the directional indicator indicates a direction the probe is to be moved to locate a conductance point with the primary probe tip.

9. The electrical probe of claim 5, wherein the directional indicator is implemented as an LCD screen.

10. The electrical probe of claim 5, wherein the directional indicator includes one or more lights associated with each of the one or more secondary probe tips.

11. The electrical probe of claim 1, further comprising a grip member that is connected to the probe and is positioned to be substantially perpendicular to the primary probe tip.

12. The electrical probe of claim 1, wherein the probe is connected to an electrical cord.

13. The electrical probe of claim 1, wherein the probe includes a battery.

14. The electrical probe of claim 1, wherein the indicator is located on the distal end of the probe.

15. The electrical probe of claim 1, wherein the indicator is a verification indicator.

16. The electrical probe of claim 1, wherein the one or more secondary probe tips are disposed around the primary probe tip at an angle increment that falls within a range of 10° to 90° increments.

17. The electrical probe of claim 1, wherein the one or more secondary probe tips are disposed around the primary probe tip at an angle increment that falls within a range of 10° to 180° increments.

18. A system comprising:
a processor that:
receives a bioelectrical reading from one or more probe tips,
determines which bioelectrical reading from the one or more probe tips indicates a highest electrical conductivity among the one or more probe tips; and
a probe comprising:
a primary probe tip,
one or more secondary probe tips,
a directional indicator, and
a verification indicator,
wherein the processor further identifies a direction of the highest electrical conductivity among the one or more secondary probe tips for the primary probe tip from the determined bioelectrical reading and indicates the identified direction by the directional indicator.

19. The system of claim 18, wherein the directional indicator comprises:
one or more light emitting diodes.

20. A method comprising:
receiving, by one or more processors, a bioelectrical reading from one or more secondary probe tips;
determining, by the one or more processors, which bioelectrical reading from the one or more secondary probe tips indicates the highest electrical conductivity;
identifying, by the one or more processors, a direction of the bioelectrical reading from the one or more probe tips for a primary probe tip;
displaying, by the one or more processors, a directional indicator associated with the identified direction.

21. The method of claim 20, wherein the receiving of the bioelectrical reading from one or more secondary probe tips occurs in response to placing one or more of the secondary probe tips in contact with person's skin.

22. The method of claim 20, wherein displaying the directional indicator includes one of illuminating a directional indicator and providing a directional indicator on a screen.
